(19) United States
(12) Patent Application Publication
(10) Pub. No.: US 2023/0393033 A1
(43) Pub. Date: Dec. 7, 2023

(54) CARTRIDGE, TRANSFER DEVICE, METHOD FOR SAMPLING AIR, AND PRODUCTION ISOLATOR SYSTEM

(71) Applicant: MERCK PATENT GMBH, DARMSTADT (DE)

(72) Inventors: Stéphane OLIVIER, Molsheim (FR); Vincent SCHAAL, Molsheim (FR)

(73) Assignee: MERCK PATENT GMBH, DARMSTADT (DE)

(21) Appl. No.: 18/033,259

(22) PCT Filed: Oct. 20, 2021
(86) PCT No.: PCT/EP2021/079029

§ 371 Date: Apr. 21, 2023

(30) Foreign Application Priority Data
Oct. 23, 2020 (EP) 20306274.0

(51) Int. Cl. G01N 1/22; C12M 1/12; C12M 3/00; C12M 1/00

(52) U.S. Cl. CPC G01N 1/2273; C12M 25/04; C12M 23/42; C12M 23/52; C12M 23/48

(57) ABSTRACT

The application relates to a cartridge for a plurality of petri-dishes, comprising a cylindrical cartridge housing configured to receive the plurality of petri-dishes in a stack aligned with an axial direction of the cartridge housing in an interior space of the cartridge housing such that the stack of petri-dishes can be moved in the axial direction within the interior space. An axial end portion of the cartridge housing has a first opening dimensioned so that the petri-dishes can be exposed from the cartridge housing by sliding in the axial direction. Another axial end portion of the cartridge housing has a second opening dimensioned to prevent removal of the petri-dishes through the second opening and so that a piston can be inserted into the interior space of the cartridge housing for moving the stack of petri-dishes (P) in the axial direction towards the first opening.

### TECHNICAL FIELD

The present application concerns a cartridge for a plurality of petri-dishes, a transfer device for a production isolator, a method for sampling air in a production isolator, and a production isolator system. The present application particularly concerns the field of testing in pharmaceutical and food processing and more particularly environmental monitoring of clean or ultra clean closed processing areas or production isolators (these terms will be used interchangeably in this specification). It is also applicable to other processing situations where cleanness of a processing area or environment is to be determined and monitored, for example in the field of semiconductor, electronics or aircraft manufacturing.

### BACKGROUND

In order to monitor environmental conditions in closed processing areas of the above type it is common practice in passive air sampling to place one or more media plate/plates in an activity zone of the production area and expose them to the surrounding air such that they can capture the maximum amount of particles in the surrounding air. Larger particles tend to settle faster on the plates due to gravitational force. Smaller particles take some time in settling due to factors such as air currents. Media plates work best in still areas. The microorganisms from the air may settle on the media plates alone or in colonies.

In active monitoring of air in closed production areas a microbial air sampler is used to force air into or onto a collection medium over a specified period of time. The collection medium can be a common petri-dish, for example including a nutrient agar-based test media or other suitable test media depending on the need.

The collection media, for example in the form of the media plates, petri-dishes or settle plates (the terms will be used interchangeably in this specification), have to be transferred repeatedly into the production area and removed therefrom for further handling and evaluation. This is commonly done in a manual process where one or more plates or petri-dishes are conveyed manually through a sterile transfer port into and from the interior of the production area. However, the manual handling of the petri-dishes involves a high risk of contamination when handling the media plates, i.e. that lids are inadvertently opened, displaced or removed from their media plates during handling involving introduction, installation and removal, thereby compromising the detection result, in particular when a plurality of them are handled in batches.

Sterile transfer ports for selective access through a valve to a clean processing area without compromising the sterility are known. Such systems are also known as “RIP” or “rapid transfer ports” and the present application is directed to a transfer device that is useful in conjunction with such a transfer port, i.e. is configured so as to be compatible with the respective valve design.

For example, GB 2237816 A1 discloses a double-door transfer port which allows a contained transfer between a container and an isolator, i.e. a clean processing area. The container is docked with its closed port and then, from within an isolator, the port door is opened. The docking of the container to the isolator may be accomplished using a bayonet system, in twisting the container around its axis to dock it into place at the port, or as in this prior art, the whole of the lid and bayonet-closure mechanism of the container are housed in a short cube- or—collar-like extension of the container mounted on the container itself via a gas-tight slip-ring joint. This type of device only presents the open container to be accessible from a lateral side from the inside of the isolator, thus making the process of handling the objects difficult, in particular where the objects to be transferred are media plates, petri-dishes or settle plates for the reasons given above.

What is desired is an at least partly or preferably fully automated process that does not involve human handling steps for introducing, sampling and removing the media plates into and from the closed processing areas or production isolators.

It is furthermore preferable to provide means for such a partly or fully automated process with which it is possible to use standard media plates available on the market, preferably the so-called petri-dishes.

The present application considers application of the transfer principle of docking a device holding one or more objects to be transferred to a rapid transfer port of an isolator and transferring the objects from the device into the isolator and vice versa.

The present application aims at providing a cartridge and a transfer device for a production isolator with which a plurality of petri-dishes, i.e. a batch can be introduced into and removed from a closed processing area or production isolator for the purposes of air sampling without compromising the sterility and without requiring a large space inside the isolator.

The present application further aims at providing a method for sampling air in a production isolator by means of a sterile and effective process, preferably with a large degree of automation.

### SUMMARY

According to the present application this object is solved by providing a cartridge for a plurality of petri-dishes as defined by claim 1, a transfer device for a production isolator as defined by claim 4, a method for sampling air in a production isolator as defined by claim 10, and a production isolator system as defined by claim 16. Preferred embodiments are defined in the dependent claims.

The present application in particular provides a cartridge for a plurality of petri-dishes, comprising a cylindrical cartridge housing configured to receive the plurality of petri-dishes in a stack aligned with an axial direction of the cartridge housing in an interior space of the cartridge housing such that the stack of petri-dishes can be moved in the axial direction within the interior space, wherein an axial end portion of the cartridge housing has a first opening dimensioned so that the petri-dishes can be exposed from the cartridge housing by the sliding motion in the axial direction, wherein another axial end portion of the cartridge housing has a second opening dimensioned to prevent removal of the petri-dishes through the second opening and so that a piston can be inserted into the interior space of the cartridge housing for moving the stack of petri-dishes in the axial direction towards the first opening.

Preferably, the cartridge further comprises a plurality of petri-dishes in a stack received in the interior space of the cartridge housing in a sterile environment.

Preferably, the cartridge further comprises a film respectively sealing the first and second openings in a removable manner.

The present application thus also provides a transfer device for a production isolator, comprising a main body configured so that a cartridge as defined herein can be loaded into an interior space of the main body, preferably in a defined angular orientation; an opening at one end portion of the main body, said opening configured to be closed by a door; and a piston arranged in the main body so that the piston can enter the second opening of the cartridge housing loaded in the interior space to move the stack of petri-dishes in the axial direction to expose the petri-dishes at the opening of the main body.

Preferably, the opening at the one end portion of the main body is configured to be selectively covered by a sieve element so that air and can flow from the outside of the transfer device towards a tray (or receptacle) of an uppermost petri-dish exposed at the opening of the main body after passing through the sieve element.

Preferably, the piston is rotatable between a first and a second rotational position, and the piston is formed to be able to enter the second opening of the cartridge housing loaded in the interior space when in the first rotational position so as to enable pushing of the stack of petri-dishes in the cartridge housing towards the one end portion of the main body, and is formed to be prevented from entering the second opening when in the second rotational position so as to enable pushing of the cartridge housing towards the one end portion of the main body.

Preferably, the transfer device further comprises a suction port communicating with the interior space of the main body.

Preferably, the transfer device further comprises a door attached to the main body to selectively open/close the opening of the main body.

Preferably, the transfer device further comprises coupling means configured to couple the opening of the main body to a transfer port; and a cartridge as defined herein loaded into the interior space of the main body.

The present application thus also provides a method for sampling air in a production isolator, comprising:
a) providing a first transfer device according to the invention so as to be coupled to a transfer port of the production isolator;
b) moving the stack of Petri-dishes by means of the piston to expose the uppermost Petri-dish of the stack at the opening at the one end portion of the main body;
c) removing a lid of the exposed petri-dish from its tray and subsequently covering the opening at the one end portion of the main body by a sieve element so that air from an inside of the production isolator can pass through the sieve element into the tray of the petri-dish exposed at the opening of the main body;
d) removing the sieve element and re-attaching the lid to the tray of the petri-dish; and
e) removing the petri-dish from the stack.

The method preferably comprises:
a2) providing a second transfer device as defined herein that is coupled to another transfer port of the production isolator; and
f) transferring, after step e), the petri-dish removed from the stack of the first transfer device to and into the second transfer device.

The method preferably comprises:
g) repeating the steps b) to f) until a predetermined number of petri-dishes have been transferred from the first transfer device into the second transfer device; and
h) removing the second transfer device from the other transfer port of the production isolator.

The present application thus also provides a method for sampling air in a production isolator, comprising:
a4) providing a cartridge with a plurality of petri-dishes as defined herein;
a5) introducing the cartridge into the production isolator;
a6) providing a transfer device as defined herein so as to be coupled to a transfer port of the production isolator;
b1) removing the film sealing the first and second openings of the cartridge in the production isolator to expose a petri-dish of the stack in the cartridge;
c1) removing a lid of the exposed petri-dish from its tray so that air from the inside of the production isolator can flow into the tray of the petri-dish exposed at the first opening of the cartridge;
d1) re-attaching the lid to the tray to the petri-dish; and
e1) transferring the petri-dish from the stack of petri-dishes of the cartridge to and into the transfer device.

The method preferably comprises:
f2) repeating the steps b1) to e1) until a predetermined number of petri-dishes have been transferred from the cartridge into the transfer device; and
g2) closing the transfer port and the door of the transfer device and removing the air sampling device from the port of the production isolator.

The method preferably comprises providing at least one transfer device by:
aa) coupling the transfer device to a transfer port of the production isolator; and
ab) opening the transfer port and the door of the first transfer device.

The present application finally also provides a production isolator system comprising a production isolator including an isolator housing configured to provide a sterile environment in an inner space of the isolator housing, and at least one transfer port formed at the production isolator for allowing a transfer into and out from the inner space of the isolator housing; at least one transfer device as defined herein; and an air sampling mechanism for sampling air in the inner space of the isolator housing, and wherein the at least one transfer port is configured to be coupled to the transfer device, and wherein the air sampling mechanism is configured to handle petri-dishes exposed at the opening at the one end portion of the main body of the transfer device so that air can flow from the inner space of the isolator housing into a tray of an exposed petri-dish.

The present application accordingly provides a solution for aseptically transferring a plurality of petri-dishes, i.e. a batch through a rapid transfer port in a controlled standardized process that is compatible with environmental monitoring in a fully automated process in a sterile environment.

The transfer device is compatible with the existing standard rapid transfer ports for an isolator and the cartridge is compatible with the standard petri-dishes or settling plates and they are capable of presenting the petri-dishes in an essentially horizontal orientation in a convenient and repeatable way for manual or automated gripping, for example by a robot gripper. Thereby, transfer into and out from the isolator can be performed in an easy, controlled and reliable manner.

Further, the petri-dishes are presented in a defined position, thereby facilitating cooperation with a gripper of an automated handling device (i.e. a robot) while still allowing a manual processing or handling if needed.

The transfer device provides a compact structure and a low footprint inside the isolator is required to present the petri-dishes for further handling.

### BRIEF DESCRIPTION OF THE DRAWINGS

Preferred embodiments will be described below by reference to the attached exemplary schematic drawings, in which:

FIG. 1 shows a transfer device for a production isolator according to an embodiment in a perspective view from the top front:

FIG. 2 shows the transfer device of FIG. 1 in a perspective cut-away view from the top front;

FIG. 3 shows a cartridge for a plurality of petri-dishes according to an embodiment in a sealed state in a perspective view from the top front;

FIG. 4 shows the cartridge of FIG. 3 in a perspective cut-away view from the bottom front;

FIG. 5 shows the cartridge of FIG. 3 and FIG. 4 in an unsealed state in a perspective view from the bottom front;

FIG. 6 shows a detail of a lower end portion of the transfer device of FIG. 1 and FIG. 2 without a cartridge in a perspective cut-away view;

FIG. 7 shows the transfer device of FIG. 1 and FIG. 2 loaded with a cartridge in a perspective cut-away view;

FIG. 8 shows the transfer device of FIG. 7 in an air sampling state in a perspective cut-away view;

FIG. 9 shows a detail of an upper axial end portion of the transfer device of FIG. 7 and FIG. 8 in the air sampling state in a cut-away view;

FIG. 10 shows a process of removing an uppermost petri-dish from a stack in a first transfer device and transferring a used petri-dish to a second transfer device;

FIG. 11 shows a transfer device according to another embodiment before coupling to a transfer port in a perspective view;

FIG. 12 shows the transfer port of FIG. 11 in a cut-away perspective view;

FIG. 13 shows the transfer device of FIG. 11 coupled or docked to the transfer port of FIG. 12 with a door of the transfer device still in a closed state in a cut-away view;

FIG. 14 shows the transfer device of FIG. 13 in the coupled or docked state with the door removed and the petri-dishes presented inside the isolator in a cut-away view;

FIG. 15 shows the transfer device and the transfer port of FIG. 13 and FIG. 14 in in an air sampling state in a cut-away view;

FIG. 16 shows a process of removing an uppermost petri-dish from a stack of a first transfer device of FIG. 11 and of transferring the petri-dish to a second transfer device in a cut-away view;

FIG. 17 shows a schematic representation of a transfer isolator coupled with a transfer device as defined herein;

FIG. 18 shows a schematic representation of a production isolator system as defined herein with two transfer devices as defined herein docked to transfer ports of the isolator and are open for illustrating the stage of the process of sampling air where petri-dishes are introduced into the isolator for air sampling;

FIG. 19 shows a schematic representation of the production isolator system of FIG. 18 in a state where the transfer devices are closed at the transfer ports immediately before the stage where the used petri-dishes are removed from the transfer port;

FIG. 20 shows a schematic representation of a transfer isolator illustrating a process of transferring cartridges as defined herein to an outside of the isolator system without using the transfer device.

### DETAILED DESCRIPTION

For the purposes of the present application, terms such as “horizontal”, “vertical”, “perpendicular”, and similar terms are—if not already explicitly indicated—considered to be “essentially horizontal”, “essentially vertical”, “essentially perpendicular”, provided that this does not negatively affect functionality. Preferably, the term “essentially” is to denote a deviation of at most 10°, more preferably of at most 5°, even more preferably of at most 4° or 3°, still even more preferably of at most 2° or 1° from being horizontal, vertical, and perpendicular, respectively.

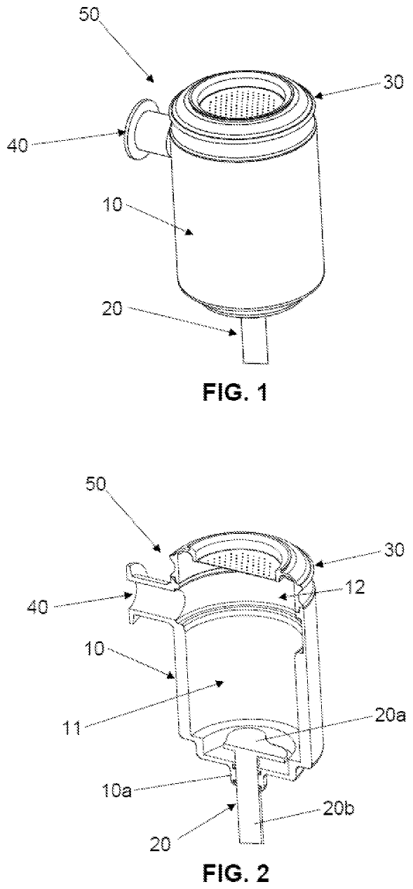

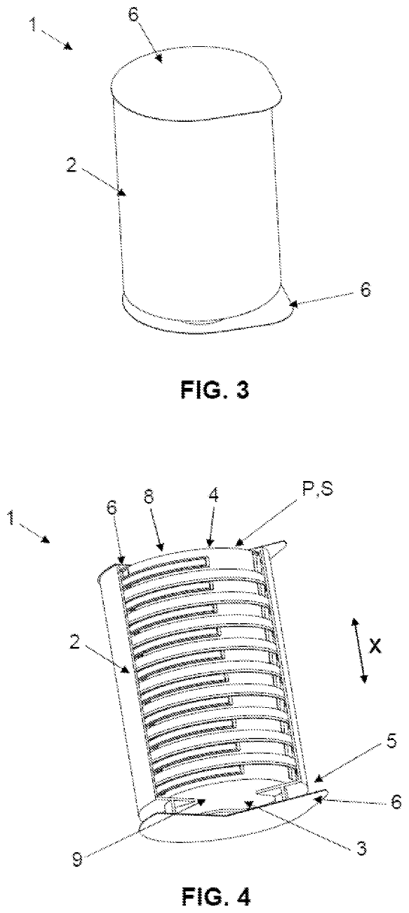

An embodiment of a transfer device 50 for a production isolator 100 as defined herein is shown in FIGS. 1, 2, and 6 to 10 and a compatible embodiment of a cartridge 1 for a plurality of petri-dishes P to be used in conjunction with the transfer device 50 is shown in the FIGS. 3, 4 and 5.

The cartridge 1 includes a cylindrical cartridge housing 2 configured to receive the plurality of petri-dishes P, i.e. a batch in a stack S aligned with an axial direction X of the cylindrical cartridge housing 2 in an interior space 3 of the cartridge housing 2. The interior space 3 is formed and dimensioned so that the stack S of petri dishes P can be moved in the axial direction X back and forth within the interior space 3. Preferably the movement is guided by a sliding contact along the inner peripheral wall of the cartridge housing 2 but guiding protrusions (not shown) raised above the inner peripheral wall of cartridge housing 2 may be provided for that purpose.

The cylindrical cartridge housing 2 has a first opening 8 at one axial end 4 (the upper end in the posture of FIG. 4) and a second opening 9 at the opposite axial end 5 as shown in FIG. 4. The first opening 8 is dimensioned so that the petri-dishes P can be exposed from the cartridge housing 2 by the sliding motion in the axial direction X. The stack of petri-dishes P thus can be moved relative to the cartridge housing 2 towards and beyond the first opening 8 to allow a sequential access to the uppermost dish in the stack or to plural petri-dishes to be used for air sampling and/or petri-dishes can be placed on the stack to be transferred back into the cartridge housing. It is preferred that the petri-dishes are placed in cartridge housing 2 with their lids facing in the direction of the first opening 8, i.e. in general to the top of the cartridge housing 2.

The second opening 9 is dimensioned to prevent removal of the petri-dishes P through the second opening 9 so that the stack S of petri-dishes can rest on the lower end of the cartridge housing. Furthermore, the second opening 9 is dimensioned so that a head of a piston 20 can be inserted through the opening into the interior space 3 for engaging with the lowermost petri-dish of the stack and pushing the stack S in the axial direction X.

In order to maintain the interior space 3 of the cartridge housing 2 and the stack S of petri dishes P in a sterile environment CA until the cartridge is used with the transfer device 50 described below, the first and second openings 8,9 can be respectively sealed by a film 6 in a removable manner. This sealed state of the cartridge 1 is shown in FIGS. 3 and 4. The film 6 may be adhered to the portions surrounding the openings or to dedicated sealing rims or it may be held in place by a connector. In addition or in an alternative the entire cartridge may be placed and sealed in a transport bag or container until use.

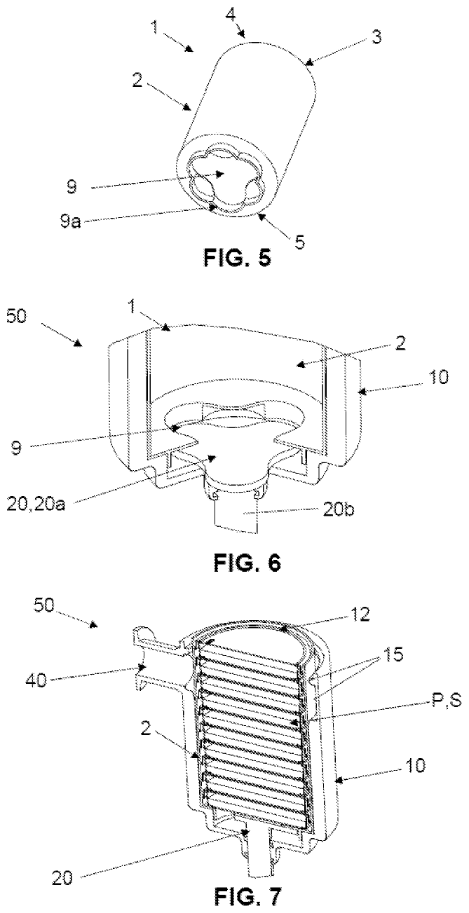

As shown in FIGS. 5 and 6, the second opening 9 may have an outline with several recesses or protrusions distributed about the circumference so as to allow insertion of a head of the piston 20, which has a complementary shape or outline with recesses or protrusions, in a specific rotational orientation where protrusions and recesses match to each other, but to prevent insertion where they do not match.

As shown in FIGS. 5 and 6 the bottom, outwardly facing side of the cartridge housing may be provided with an axially protruding rim or rib 9a around the second opening 9 with protrusions and recesses matching at least partly those of the head of the piston but relatively rotated from those of the second opening 9 to receive the head in a defined rotational position where it cannot enter the opening 9. In this state the movement of the piston in the axial direction will engage the head of the piston with the outside surface of the housing and will move the entire cartridge in the axial direction towards an opening of a transfer device 50 described later. The protruding rim or rib 9a has a function of placing the piston 20, when the same is rotated, in the two defined rotational positions, i.e. the first rotational position where the piston 20 bears against the lower end portion 5 of the cartridge housing 2 to lift the entire cartridge housing or the second rotational position where it may enter the second opening 9 and letting it pass so that it may bear against the lowermost petri-dish in the cartridge housing 2 to move the petri-dish (and all the others in a stack) in the interior space 3 of the cartridge housing towards the first opening 8. The rim or rib does not have to be continuous but can be made up from several spaced apart segments.

The form of the second opening 9 and of the head of the piston 20 is not limited to any particular form or to a specific number of protrusions and recesses and they need not be complementary as long as the two distinct rotational positions of the piston allowing selective lifting of the cartridge housing or the stack of petri-dishes is possible.

The transfer device 50 for a production isolator 100 according to an embodiment comprises a cylindrical main body 10, an interior space 11 configured to receive and accommodate a cartridge 1 as defined herein so that the cartridge 1 can be moved in the axial direction within the interior space 11, an opening 12 at an end portion of the main body 10, and a piston 20 provided at an end portion of the main body that is opposite to the opening 12.

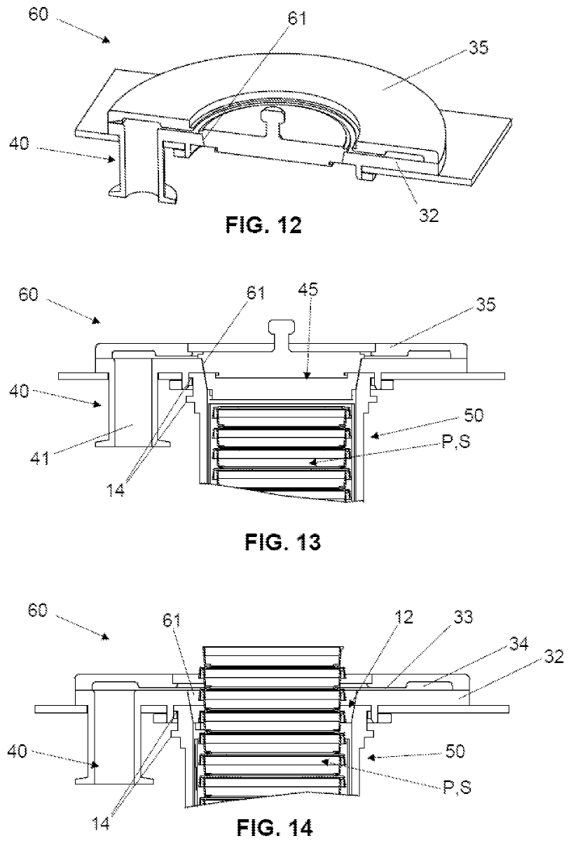

The opening 12 is located at an axial end portion of the main body 10 (the upper portion in the posture of FIG. 2) and is configured to be selectively closed by a door 45 allowing to seal the interior space 11 against the outside environment (see FIG. 11 in combination with FIG. 2). The main body is provided with coupling means 14 configured to couple the opening 12 and the door 45 to a rapid transfer port 60 and open or close the door 45 in the coupled state to create a sterile communication between the interior space 11 of main body 10 of the transfer device 50 and the interior space or clean environment CA of the isolator 100 that is sealed at all stages from the outside environment GA (see FIGS. 11 to 20). The coupling means 14 can be formed, for example, by a bayonet coupling arrangement as shown in FIGS. 11 to 15 but can also be a threaded coupling arrangement.

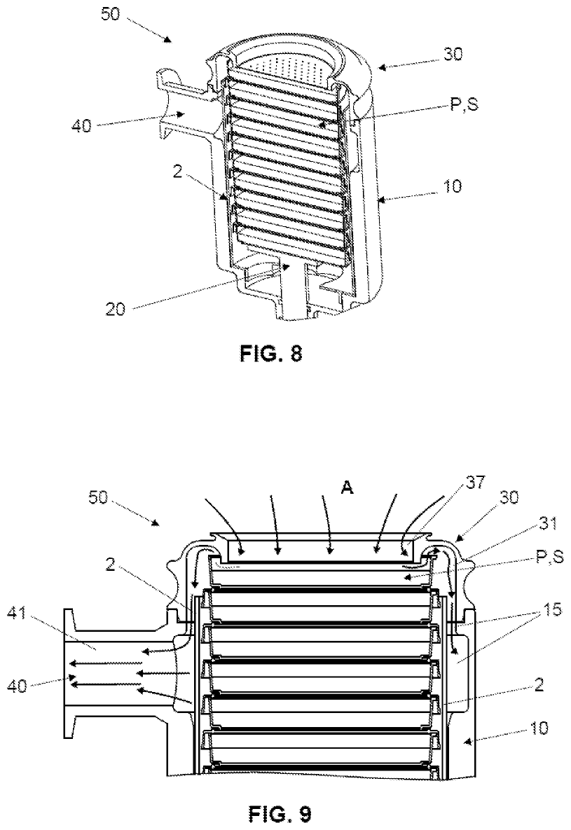

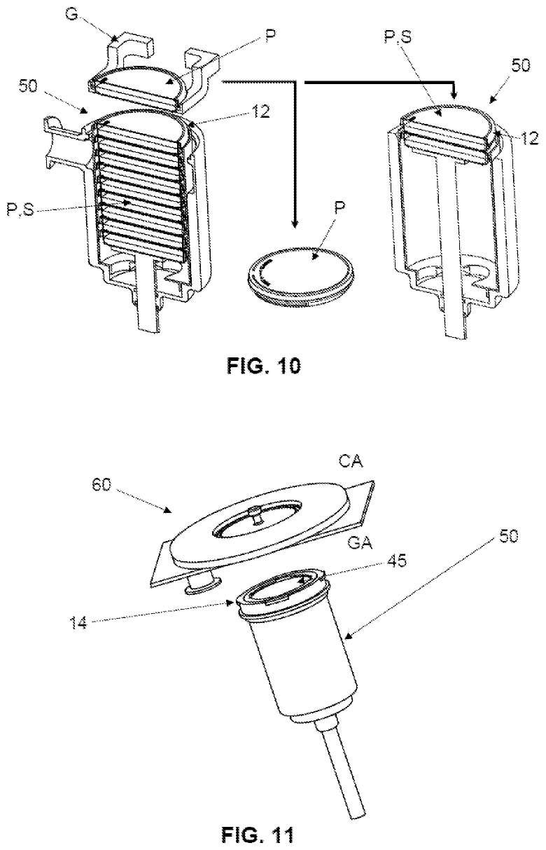

The piston 20 is arranged at a lower end portion of the main body 10 so that the head of the piston 20 is located inside the interior space 11 and a shaft 20b of the piston is guided to the outside through a sealed port 10a that allows a sliding movement of the piston in the interior space 11 while maintaining the sealed state. The head 20a of the piston 20 is formed as described above so that, depending on the rotational position, it can either enter the second opening 9 of the cartridge housing 2 loaded into the interior space 11 to push the stack of petri-dishes towards the first opening 8 of the cartridge and the opening 12 of the main body 10 of the transfer device 50 to expose the petri-dishes P outside the opening 12 (see FIGS. 8 and 10) or bear against the lower end of the cartridge 1 to push the entire cartridge towards the opening 12 of the transfer device 50 (see FIG. 7).

Once one or several uppermost petri-dish/dishes P of the stacks S is/are presented outside the opening 12 above the top edge of the main body, it/they can be gripped individually or in batches by hand or by a gripper G and/or an air sampling mechanism 110 or put back on top of a stack of (another) transfer device 50 for eventual removal from the interior space or clean environment CA of the isolator 100 after the testing cycle is completed (see FIG. 10).

If the air sampling is to be carried out directly at the transfer device 50 the opening 12 at the end portion of the main body 10 is configured to be removably covered by a sieve element 30 after the transfer device 50 is docked to the transfer port of the isolator 100, the door 45 has been opened, the stack of petri-dishes P has been pushed out from the cartridge until the uppermost petri-dish is raised above a top edge of an opening of the transfer port (not shown), and a lid of the uppermost petri-dish P in the stack S has been removed (for example manually by a user or automatically by means of a gripper G) to expose the inside of the tray P2. The sieve element 30 surrounds the outer periphery of the transfer device 50 around its opening 12 to prevent any flow of air into the transfer device 50 except through an inlet opening 37 of the sieve element and the sieve element 30 needs to be removed prior to allowing lifting of the lid. In this way air A can pass only through the inlet opening 37 and further through pores of the sieve element 30 from the clean environment CA of the isolator 100 and is directed to flow towards the tray P2 of the uppermost petri-dish P exposed at the opening 12 at the upper portion of the main body 10. This air sampling process is shown in FIG. 9. The sieve 30 is configured to guide a stream of the air A into the tray P2 to project the air in contact with a collection medium (for example an agar medium) in the petri-dish P for air sampling. From the dish the air flows to the outer periphery where it enters into an annular channel 31 surrounding the sieve 30.

To discharge the air to the outside the transfer device 50 comprises an outlet port 40 extending from a lateral side of the main body and having a flow passage communicating with the interior space 11 of the main body 10. The outlet port 40 may be connected to an external vacuum pump (not shown) to create a reduced pressure on the downstream side and induce the stream of the air. The flow passage of the outlet port 40 connects, towards the upstream side, to a circumferential annular recess formed in the inner peripheral wall of the end portion of the main body 10 and communicating with the annular channel 31 surrounding the sieve 30. The circumferential annular recess 15 is separated from the interior space 3 of the cartridge holding the petri-dishes by the outer circumferential wall of the cartridge housing 2. Thus, as shown in FIG. 9, the stream of the air A is guided, after flowing towards and along the collection medium in the uppermost tray P2, to the circumferential side of the tray P2, through the annular channel 31 surrounding the sieve 30, through the circumferential annular recess 15 formed in the inner peripheral wall of the main body and out through the flow passage in the suction port 40.

The outlet port 40 does not necessarily have to be a part of the transfer device 50 but can be provided as a part of the transfer port 60 as shown in FIGS. 11 to 16. In this embodiment the transfer port 60 includes an annular plate 32 surrounding a port opening 61 and including a circumferential flow channel 33, 34 serving as air collection channel and leading from an entry portion near the edge of the port opening 61 to an outlet port 40 which also may be connected to an external vacuum pump (not shown) to create a reduced pressure on the downstream side and induce the stream of the air. The flow channel 33, 34 may be formed as a hollow passage inside the annular plate 32 or may be formed by attaching an annular flange 35 to the annular plate 32 to close grooves in the plate and/or the flange to form the air collection channel.

A valve mechanism (not shown) may be provided to open the air collection channel to the outlet port 40 only when the transfer device 50 is docked to the rapid transfer port and the transfer port is opened. The valve mechanism may include the door 45 of the transfer device 50 and/or the door of the transfer port sealing air collection channel towards the interior space of the isolator as long as they are in the closed positions.

In this case, too, the opening 12 at the end portion of the main body 10 is configured to be removably covered by a sieve element 30 after the transfer device 50 is docked to the transfer port of the isolator 100, the door 45 has been opened, the stack of petri-dishes P has been pushed out from the cartridge until the uppermost petri-dish is raised above a top edge of an opening 61 of the transfer port 60, and a lid of the uppermost petri-dish P in the stack S has been removed (for example manually by a user or automatically by means of a gripper G) to expose the inside of the tray P2. The sieve element 30 surrounds the outer periphery of the transfer device 50 around its opening 12 to prevent any flow of air into the transfer device 50 except through an inlet opening 37 of the sieve element 30.

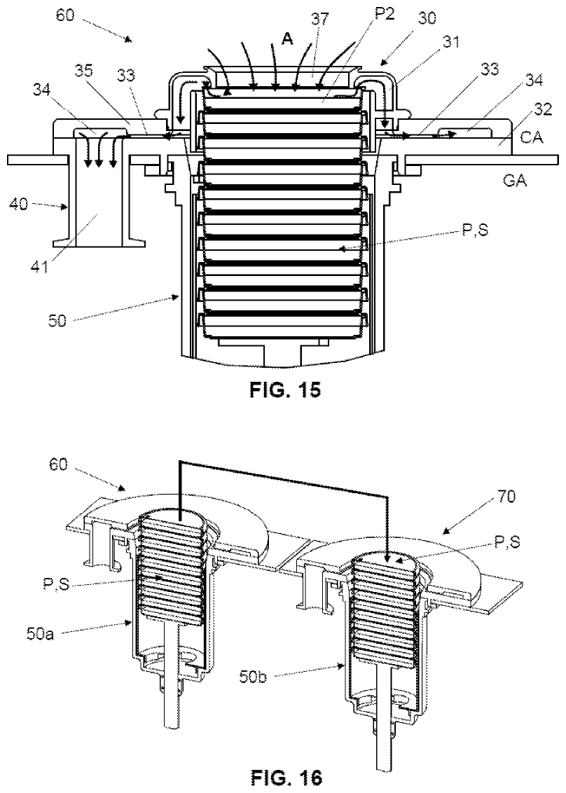

The sieve 30 is configured to guide a stream of the air A into the tray P2 to project the air in contact with a collection medium (for example an agar medium) in the petri-dish P for air sampling. From the dish the air flows to the outer periphery where it enters into an annular channel 31 surrounding the sieve 30. The air collection channel 33, 34 of the annular plate 32 and flange 35 connects, towards the upstream side, to the annular channel 31 surrounding the sieve 30, and to the outlet port 40 towards the downstream side (see FIG. 15).

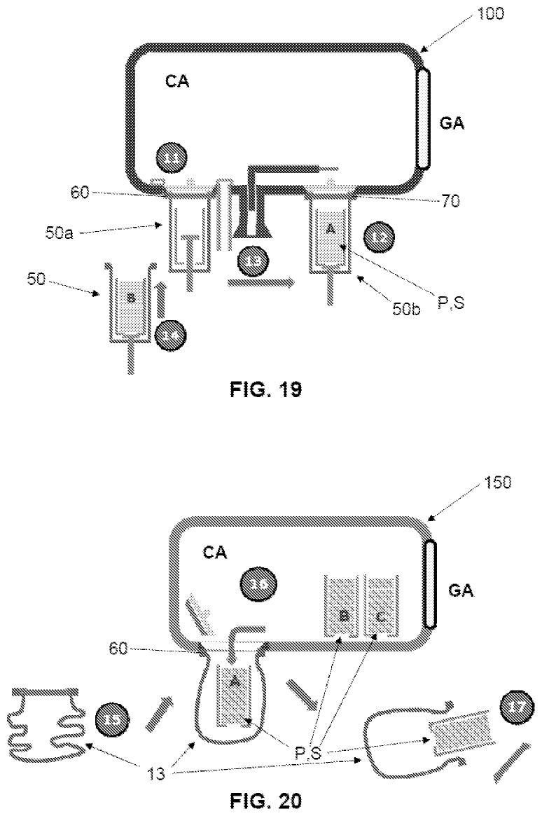

An embodiment of the method for sampling air A in a production isolator 100 using the transfer device and cartridge as defined herein is shown in FIGS. 18 and 19. The method comprises:
a) providing the above described transfer device 50 with a stack of petri-dishes in a sterile state as a first transfer device 50a at a transfer port 60 of the production isolator 100;
b) pushing the stack S of petri-dishes P in the essentially upward vertical direction by means of the piston 20 to present and expose the uppermost petri-dish P of the stack S at the opening 12 at the upper portion of the main body 10, indicated as step 8) in FIG. 18;
c) removing a lid P1 of the exposed uppermost petri-dish P from its tray P2 and subsequently covering the opening 12 at the upper portion of the main body 10 by a sieve element 30 so that air A from an inside of the production isolator 100 (i.e. from the clean atmosphere CA) can pass through the sieve element 30 and can flow towards the tray P12 of the uppermost petri-dish P exposed at the opening 12 of the main body indicated as step 9) in FIG. 18;
d) removing the sieve element 30 and re-attaching the lid P1 to the tray P2 of the uppermost petri-dish P to close it; and
e) removing the uppermost Petri-dish P from the stack S indicated as step 10) in FIG. 18.

Preferably, steps a) to e) are conducted in sequence a), b), c), d) and e).

Preferably, the method for sampling air A in the production isolator 100 further comprises:
a2) providing the above described transfer device as a second transfer device 50b that has an empty cartridge at another transfer port 70 of the production isolator 100, as shown in FIGS. 18 and 19; and
f) transferring, after opening the transfer port and after step e), the petri-dish P removed from the stack S of the first transfer device 50 to and into the cartridge of the second transfer device 50, indicated as a part of the step 10) in FIG. 18.

Step a2) may be performed at any stage in the process as long as it is performed before step f).

In a preferred embodiment the method further comprises:
g) repeating the steps b) to f) until a predetermined number (or all) of petri-dishes P have been transferred from the first transfer device 50 (which is then empty) into the second transfer device 50 (which is then full); and
h) closing the other transfer port and removing the second transfer device 50 from the other transfer port 70 of the production isolator 100, indicated as step 12) in FIG. 19. The empty first transfer device 50a may be removed as well after closing the transfer port 60 or it may be used to receive the petri-dishes from another fresh transfer device that is coupled to the other transfer port 70 in the next step.

Preferably, steps g) and h) are performed in this sequence.

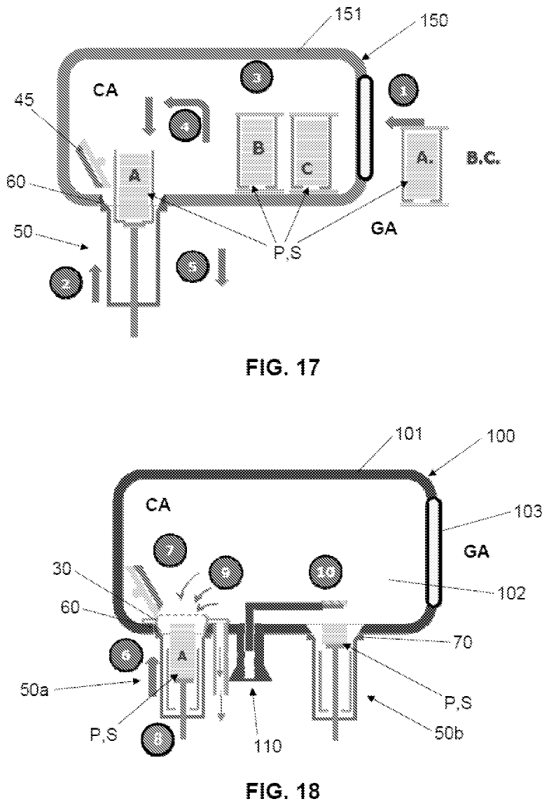

FIG. 17 shows a schematic representation of a transfer isolator 150 coupled with a transfer device 50 for illustrating a preparation process using the transfer device 50 and the cartridge 1 as defined herein.

A transfer isolator 150 differs from a production isolator 100 in that the transfer isolator 150 does not necessarily have installations or equipment for production but can only be used for storage or preparation of the petri-dishes in batches for the use in the air testing of a production isolator. The transfer isolator 150 includes an isolator housing 151 configured to provide a sterile environment CA in an inner space of the isolator housing 151 and one or more transfer ports 60 formed in the isolator housing 151 for transfer between the inner space and an outside of the isolator housing 101.

The transfer isolator 150 can be used for a preparation, i.e. the loading or unloading of transfer devices 50 with batches of fresh or spent petri-dishes in the cartridges. The preparation process comprises
introducing the cartridge 1 as defined herein with the plurality of petri-dishes P into the transfer isolator 100, indicated as step 1) in FIG. 17;
providing the transfer device 50 as defined herein at a transfer port 60 of the transfer isolator 150, indicated as step 2) in FIG. 17;
removing the film 6 sealing the first and second openings 8,9 of the cartridge 1 in the transfer isolator 150, indicated as step 3) in FIG. 17; and
transferring the unsealed cartridge 1 with the stack S of petri-dishes P to and into the transfer device 50, indicated as step 4) in FIG. 17.

After this preparation process the loaded transfer device 50 can be decoupled from the transfer isolator, indicated as step 5) in FIG. 17, and transferred and coupled to the transfer port 60 of the production isolator 100.

Another method for sampling air A in a production isolator 100 according to an embodiment combines substantially the above described preparation process with an air sampling process in the production isolator 100. The method comprises:
a4) providing the cartridge 1 as defined herein with a plurality of petri-dishes P;
a5) introducing the cartridge 1 with the plurality of petri-dishes P into the production isolator 100, indicated as step 1) using a transfer isolator 150 in FIG. 17 as an example;
a6) providing a transfer device 50 as defined herein that has an empty cartridge at a transfer port 60) of the production isolator 100, indicated as step 2) using the transfer isolator 150 in FIG. 17 as an example;
b1) removing the film 6 sealing the first and second openings 8,9 of the cartridge 1 in the production isolator 100 to expose the uppermost petri-dish P of the stack S in the cartridge 1, indicated as step 3) for a transfer isolator 150 in FIG. 17 as an example;
c1) removing a lid P1 of the exposed petri-dish P from its tray P2 so that air A from the inside of the production isolator 100 can flow towards the tray P2 of the uppermost petri-dish P exposed at the first opening 8 of the cartridge 1;
d1) re-attaching the lid P1 to the tray P2 of the uppermost petri-dish P; and
e1) transferring the petri-dish P from the stack S of petri-dishes P of the cartridge to and into the transfer device 50.

Preferably, this method for sampling air A in the production isolator 100 comprises:
f2) repeating the steps b1) to e1) until a predetermined number of petri-dishes P have been transferred from the cartridge 1 into the transfer device 50; and
g2) closing the transfer port 60 and the door 45 of the transfer device 50 and removing the transfer device 50 from the port 60 of the production isolator 100.

In a preferred embodiment, all above described methods for sampling air A in a production isolator 100 can further provide the at least one transfer device 50 at the respective transfer port by:
aa) coupling the transfer device 50 to a transfer port 60,70 of the production isolator 100; and
ab) opening the transfer port 60 and the door 45 of the first transfer device 50.

A production isolator system according to an embodiment comprises a production isolator 100 with an isolator housing 101 and at least one transfer port 60, at least one transfer device 50, and an air sampling mechanism 110 for sampling air A.

The isolator housing 101 is configured to provide a sterile environment CA in an inner space 102 of the isolator housing 101 allowing to ensure a sterile production environment in the inner space 102. The one or more transfer ports 60 formed in the isolator housing 101 is/are configured for transferring objects, in particular petri-dishes, between the inner space 102 and an outside of the isolator housing 101 allowing a sterile transfer. The one or more sampling devices 50 are the sampling devices 50 as defined herein. The one or more transfer ports 60 are configured to be coupled to the transfer device 50 with the opening 12 at the upper portion of the main body 10 of the transfer device 50.

The air sampling mechanism 110 for sampling air A in the inner space 102 by using the transfer device 50 is configured to handle petri-dishes P exposed at the opening 12 at the upper portion of the main body 10 of the transfer device 50 by opening the respective lid and attaching a sieve element as described above so that air A can flow from the inner space 102 towards a tray P2 of an petri-dish P.

When all the stacks S of petri-dishes P have been handled, the user can open the transfer isolator 150 and remove the cartridges 1 with the respective stacks S of used petri-dishes P for further analysis/incubation. If desired, the analysis can be initiated without waiting until all batches have been processed. In order to remove the used petri-dishes P for further processing from the transfer isolator 150, a simple rapid transfer port bag 13 can be used and docked to the transfer port 60 of the transfer isolator 150 as shown in FIG. 20.

1. A cartridge (1) for a plurality of petri-dishes (P), comprising:
a cylindrical cartridge housing (2) configured to receive the plurality of petri-dishes (P) in a stack (S) aligned with an axial direction (X) of the cartridge housing (2) in an interior space (3) of the cartridge housing (2) such that the stack (S) of petri-dishes (P) can be moved in the axial direction (X) within the interior space (3),
wherein an axial end portion (4) of the cartridge housing (2) has a first opening (8) dimensioned so that the petri-dishes (P) can be exposed from the cartridge housing (2) by the sliding motion in the axial direction (X),
wherein another axial end portion (5) of the cartridge housing (2) has a second opening (9) dimensioned to prevent removal of the petri-dishes (P) through the second opening (9) and so that a piston (20) can be inserted into the interior space (3) of the cartridge housing (2) for moving the stack (S) of petri-dishes (P) in the axial direction (X) towards the first opening (8).

2. The cartridge (1) according to claim 1, further comprising a plurality of petri-dishes (P) in a stack (S) received in the interior space (3) of the cartridge housing (2) in a sterile environment (CA).

3. The cartridge (1) according to claim 2, further comprising a film (6) respectively sealing the first and second openings (8,9) in a removable manner.

4. A transfer device (50) for a production isolator (100), comprising:
a main body (10) configured so that a cartridge (1) according to claim 2 can be loaded into an interior space (11) of the main body (10);
an opening (12) at one end portion of the main body (10), said opening (12) configured to be closed by a door (45); and
a piston (20) arranged in the main body (10) so that the piston (20) can enter the second opening (9) of the cartridge housing (2) loaded in the interior space (11) to move the stack (S) of petri-dishes (P) in the axial direction (X) to expose the petri-dishes (P) at the opening (12) of the main body (10).

5. The transfer device (50) according to claim 4, wherein the opening (12) at the one end portion of the main body (10) is configured to be selectively covered by a sieve element (30) so that air (A) can flow from the outside of the transfer device (50) towards a tray (P2) of an uppermost petri-dish (P) exposed at the opening (12) of the main body (10) after passing through the sieve element (30).

6. The transfer device (50) according to claim 4, wherein the piston (20) is rotatable between a first and a second rotational position, and wherein the piston (20) is formed to be able to enter the second opening (9) of the cartridge housing (2) loaded in the interior space (11) when in the first rotational position so as to enable pushing of the stack (S) of petri-dishes (P) in the cartridge housing (2) towards the one end portion of the main body (10), and is formed to be prevented from entering the second opening (9) when in the second rotational position so as to enable pushing of the cartridge housing (2) towards the one end portion of the main body (10).

7. The transfer device (50) according to claim 4, further comprising a suction port (40) communicating with the interior space (11) of the main body (10).

8. The transfer device (50) according to claim 4, further comprising a door (45) attached to the main body (10) to selectively open/dose the opening (12) of the main body (10).

9. The transfer device (50) according to claim 8, further comprising:
coupling means (13) configured to couple the opening (12) of the main body (10) to a transfer port (60); and
the cartridge (1) loaded into the interior space (11) of the main body (10).

10. A method for sampling air (A) in a production isolator (100), comprising:
a) providing a first transfer device (50; 50a) according to claim 9 so as to be coupled to a transfer port (60) of the production isolator (100);
b) moving the stack (S) of Petri-dishes (P) by means of the piston (20) to expose the uppermost Petri-dish (P) of the stack (S) at the opening (12) at the one end portion of the main body (10);
c) removing a lid (P1) of the exposed petri-dish (P) from its tray (1′2) and subsequently covering the opening (12) at the one end portion of the main body (10) by a sieve element (30) so that air (A) from an inside of the production isolator (100) can pass through the sieve element (30) into the tray (P1) of the petri-dish (P) exposed at the opening (12) of the main body (10);
d) removing the sieve element (30) and re-attaching the lid (P1) to the tray (P2) of the petri-dish (P); and
e) removing the petri-dish (P) from the stack (S).

11. The method for sampling air (A) in a production isolator (100) according to claim 10,
a2) wherein in step a) additionally providing a second transfer device (50; 50b) comprising:
a main body (10) configured so that a cartridge (1) can be loaded into an interior space (11) of the main body (10) wherein said cartridge (1) comprises:
a cylindrical cartridge housing (2) configured to receive the plurality of petri-dishes (P) in a stack (S) aligned with an axial direction (X) of the cartridge housing (2) in an interior space (3) of the cartridge housing (2) such that the stack (S) of petri-dishes (P) can be moved in the axial direction (X) within the interior space (3),
wherein an axial end portion 4) of the cartridge housing (2) has a first opening (8) dimensioned so that the petri-dishes (P) can be exposed from the cartridge housing (2) by the sliding motion in the axial direction (X),
wherein another axial end portion (5) of the cartridge housing (2) has a second opening (9) dimensioned to prevent removal of the petri-dishes (P) through the second opening (9) and so that a piston (20) can be inserted into the interior space (3) of the cartridge housing (2) for moving the stack (S) of petri-dishes (P) in the axial direction (X) towards the first opening (8), and
a plurality of petri-dishes (P) in a stack (S received in the interior space (3) of the cartridge housing (2) in a sterile environment (CA);
an opening (12) at one end portion of the main body (10), said opening (12) configured to be closed by a door (45); and
a piston (20) arranged in the main body (10) so that the piston (20) can enter the second opening (9) of the cartridge housing (2) loaded in the interior space (11) to move the stack (S) of petri-dishes (P) in the axial direction (X) to expose the petri-dishes (P) at the opening (12) of the main body (10)
wherein said second transfer device is coupled to another transfer port (70) of the production isolator (100); and additionally comprising
f) transferring, after step e), the petri-dish (P) removed from the stack (S) of the first transfer device (50; 50a) to and into the second transfer device (50; 50b).

12. The method for sampling air (A) in a production isolator (100) according to claim 11, comprising:
f) repeating the steps b) to f) until a predetermined number of petri-dishes (P) have been transferred from the first transfer device (50; 50a) into the second transfer device (50; 50b); and additionally comprising
g) removing the second transfer device (50; 50b) from the other transfer port (70) of the production isolator (100).

13. A method for sampling air (A) in a production isolator (100), comprising:
a4) providing a cartridge (1) with a plurality of petri-dishes (1′) configured so that a cartridge (1) according to claim 2 can be loaded into an interior space (11) of the main body (10) wherein cartridge (1) comprises:
a cylindrical cartridge housing (2) configured to receive the plurality of petri-dishes (P) in a stack (S) aligned with an axial direction (X) of the cartridge housing (2) in an interior space (3) of the cartridge housing (2) such that the stack (S) of petri-dishes (P) can be moved in the axial direction (X) within the interior space (3),
wherein an axial end portion (4) of the cartridge housing (2) has a first opening (8) dimensioned so that the petri-dishes (P) can be exposed from the cartridge housing (2) by the sliding motion in the axial direction (X),
wherein another axial end portion (5) of the cartridge housing (2) has a second opening (9) dimensioned to prevent removal of the petri-dishes (P) through the second opening (9) and so that a piston (20) can be inserted into the interior space (3) of the cartridge housing (2) for moving the stack (S) of Petri-dishes (P) in the axial direction (X) towards the first opening (8), and
a plurality of petri-dishes (P) in a stack (S) received in the interior space (3) of the cartridge housing (2) in a sterile environment (CA), and
further comprising a film (6) respectively sealing the first and second openings (8,9) in a removable manner,
a5) introducing the cartridge (1) into the production isolator (100);
a6) providing a transfer device (50) wherein said transfer device (50) comprises:
a main body (10) configured so that a cartridge (1) according to claim 2 can be loaded into an interior space (11) of the main body (10) wherein said cartridge (1) comprises:
a cylindrical cartridge housing (2) configured to receive the plurality of petri-dishes (P) in a stack (S) aligned with an axial direction (X) of the cartridge housing (2) in an interior space (3) of the cartridge housing (2) such that the stack (S) of petri-dishes (P) can be moved in the axial direction (X) within the interior space (3),
wherein an axial end portion (4) of the cartridge housing (2) has a first opening (8) dimensioned so that the petri-dishes (P) can be exposed from the cartridge housing (2) by the sliding motion in the axial direction (X),
wherein another axial end portion (5) of the cartridge housing (2) has a second opening (9) dimensioned to prevent removal of the petri-dishes (P) through the second opening (9) and so that a piston (20) can be inserted into the interior space (3) of the cartridge housing (2) for moving the stack (S) of petri-dishes (P) in the axial direction (X) towards the first opening (8), and
a plurality of petri-dishes (P) in a stack (S) received in the interior space (3) of the cartridge housing (2) in a sterile environment (CA);
an opening (12) at one end portion of the main body (10) said opening (12) configured to be closed by a door (45); and
a piston (20) arranged in the main body (10) so that the piston (20) can enter the second opening (9) of the cartridge housing (2 loaded in the interior space (11) to move the stack (S) of petri-dishes (P) in the axial direction (X) to expose the petri-dishes (P) at the opening (12) of the main body (10) so as to be coupled to a transfer port (60) of the production isolator (100);
b1) removing the film (6) sealing the first and second openings (8,9) of the cartridge (1) in the production isolator (100) to expose a petri-dish (P) of the stack (S) in the cartridge (1);
c1) removing a lid (P1) of the exposed petri-dish (P) from its tray (P2) so that air (A) from the inside of the production isolator (100) can flow into the tray (P2) of the petri-dish (P) exposed at the first opening (8) of the cartridge (1);
d1) re-attaching the lid (P1) to the tray (P2) to the petri-dish (P); and
transferring the petri-dish (P) from the stack (S) of petri-dishes (P) of cartridge to and into the transfer device (50).

14. The method for sampling air (A) in a production isolator (100) according to claim 13, further comprising:
f2) repeating the steps b1) to e1) until a predetermined number of petri-dishes (P) have been transferred from the cartridge (1) into the transfer device (50); and
g2) closing the transfer port (60) and the door (45) of the transfer device (50) and removing the air sampling device (50) from the port (60) of the production isolator (100).

15. The method for sampling air (A) in a production isolator (100) according to claim 10, further comprising:
providing at least one transfer device (50) by:
aa) coupling the transfer device (50) to a transfer port (60,7) of the production isolator (100); and
ab) opening the transfer port (60) and the door (45) of the first transfer device (50).

16. A production isolator system comprising:
a production isolator (100) comprising
an isolator housing (101) configured to provide a sterile environment (CA) in an inner space (102) of the isolator housing (101), and
at least one transfer port (60) formed at the production isolator (100) for allowing a transfer into and out from the inner space (102) of the isolator housing (101);
at least one transfer device (50) according to claim 4; and
an air sampling mechanism (110) for sampling air (A) in the inner space (102) of the isolator housing (101), and
wherein the at least one transfer port (60) is configured to be coupled to the transfer device (50), and
wherein the air sampling mechanism (110) is configured to handle petri-dishes (P) exposed at the opening (12) at the one end portion of the main body (10) of the transfer device (50) so that air (A) can flow from the inner space (102) of the isolator housing (101) into a tray (P2) of an exposed petri-dish (P).
